(19) United States
(12) Patent Application Publication
(10) Pub. No.: US 2021/0386826 A1
(43) Pub. Date: Dec. 16, 2021

(54) Combination for Treating Cancer

(71) Applicants: Pfizer Inc., New York, NY (US); MERCK PATENT GMBH, Darmstadt (DE); NEKTAR THERAPEUTICS, San Francisco, CA (US); Astellas Pharma Inc., Tokyo (JP)

(72) Inventors: Christoffel Hendrik Boshoff, New York, NY (US); Rossano Cesari, Roma (IT); Cristian Massacesi, Summit, NJ (US); Deborah Charych, Albany, CA (US)

(73) Assignees: Pfizer Inc., New York, NY (US); MERCK PATENT GMBH, Darmstadt, NY (DE); NEKTAR THERAPEUTICS, San Francisco, CA (US); Astellas Pharma Inc., Tokyo, CA (JP)

(21) Appl. No.: 17/291,047

(22) PCT Filed: Nov. 4, 2019
(86) PCT No.: PCT/IB2019/059459

§ 371 Date: May 4, 2021

Related U.S. Application Data
(60) Provisional application No. 62/882,424, filed on Aug. 2, 2019.
(60) Provisional application No. 62/755,944, filed on Nov. 5, 2018.

(51) Int. Cl. A61K 38/20; A61K 47/60; A61K 39/395; A61K 31/5025; A61P 35/00

(52) U.S. Cl. CPC A61K 38/2013; A61K 47/60; A61P 35/00; A61K 31/5025; A61K 39/3955

(57) ABSTRACT

Provided herein are methods and combinations for treating a subject having cancer by administering to the subject a PD-1/PD-L1 axis inhibitor, a CD-122-biased cytokine agonist, and an anti-androgen or a pharmaceutically acceptable salt thereof.

### FIELD

The instant application relates to cancer therapy. Certain embodiments relate to the treatment of an individual having cancer by administering to the individual a combination of a PD-1 axis binding antagonist with a CD-122-biased cytokine agonist, and an anti-androgen, or a pharmaceutically acceptable salt thereof.

### BACKGROUND

PD-L1 is overexpressed in many cancers and is often associated with poor prognosis (Okazaki T et al., Intern. Immun. 2007 19(7):813) (Thompson R H et al., Cancer Res 2006, 66(7):3381). Interestingly, the majority of tumor infiltrating T lymphocytes predominantly express PD-1, in contrast to T lymphocytes in normal tissues and peripheral blood. PD-1 on tumor-reactive T cells can contribute to impaired antitumor immune responses (Ahmadzadeh et al, Blood 2009 1 14(8): 1537). This may be due to exploitation of PD-L1 signaling mediated by PD-L1 expressing tumor cells interacting with PD-1 expressing T cells to result in attenuation of T cell activation and evasion of immune surveillance (Sharpe et al., Nat Rev 2002) (Keir M E et al., 2008 Annu. Rev. Immunol. 26:677). Therefore, inhibition of the PD-L1/PD-1 interaction may enhance CD8+ T cell-mediated killing of tumors.

The inhibition of PD-1 axis signaling through its direct ligands (e.g., PD-L1, PD-L2) has been proposed as a means to enhance T cell immunity for the treatment of cancer (e.g., tumor immunity). Moreover, similar enhancements to T cell immunity have been observed by inhibiting the binding of PD-L1 to the binding partner B7-1. There are currently at least five PD-1 axis binding antagonists approved by the FDA in more than 10 cancer indications (A Ribas et al, Science, 359, 1350-1355, 2018). Among these, nivolumab (OPDIVO®), and pembrolizumab (KEYTRUDA®) are each anti-PD-1 antibodies, while avelumab (BAVENCIO®), atezolizumab (TECENTRIQ®)) and durvalumab (IMFINZI®)) are each anti-PD-L1 antibodies.

The interleukin-2 receptor (IL-2R) is a heterotrimeric protein expressed on the surface of certain immune cells, such as lymphocytes, that binds and responds to the IL-2 cytokine. The IL-2 receptor is made up of 3 subunits—IL-2Rα, IL-2Rβ, and IL-2Rγ, with each of IL-2Rα and IL-2Rβ having binding affinity for IL-2 while IL-2Ry alone has no appreciable affinity. Thèze et al. (1994) Immunol. Today 17(10):481-486. Further, the IL-2Rαβ heterodimer has a faster association rate and a slower dissociation rate when binding IL-2 versus either chain alone. Liparoto et al. J. Mol. Recognit. 12(5):316-321.

CD8+ memory T-cells, which are responsible for enhancing the immune response, preferentially express the IL-2Rβ form of the IL-2R (this form of the IL-2R is also known as CD-122). Thus, administration of compounds that are CD-122-biased cytokine agonists can be expected to enhance the immune response (by, e.g., increasing the proliferation of CD8+ memory T-cells).

Thus, the art recognizes the potential of administration of IL-2Rβ-selective agonists (also known as CD-122-biased cytokine agonists) in the treatment of patients suffering from cancer.

Androgen receptor (AR) is a member of the nuclear hormone receptor family activated by androgens such as dihydrotestosterone (DHT). AR is a prime therapeutic target for treating prostate cancer. Several compounds have been developed as chemotherapy for prostate cancer. However, these compounds bind AR with affinities comparable to or less than the endogenous hormone and over time patients develop resistance to these drugs. Higher affinity and/or slower off-rate ligands (e.g. covalent ligands) are needed to provide more effective therapies.

Anti-androgens are thought to suppress androgen activity by a number of different mechanisms. One example of an anti-androgen approved for the treatment of metastatic castration-resistant prostate cancer and metastatic high risk castration sensitive prostate cancer is abiraterone acetate (marketed as Zytiga™), a steroidal CY17A1 inhibitor which is dosed in conjunction with prednisone. One specific class of anti-androgens are androgen receptor inhibitors, also known as androgen receptor antagonists, which are thought to compete with endogenous ligands, androgens, for the androgen receptor. When an antagonist binds to an androgen receptor it is thought to induce a conformational change in the receptor itself that impedes transcription of key androgen regulated genes and therefore inhibits the biological effects of the androgens themselves, such as testosterone and dihydrotestosterone. Current drugs for prostate cancer include flutamide, bicalutamide, nilutamide, enzalutamide and apalutamide. However, despite treatment with anti-androgens, for some subjects, their cancer will relapse or the subjects may develop therapeutic resistance. The mechanisms that underlie such resistance are, to date, not yet fully understood.

The combination therapy of a PD-1 axis binding antagonist with one or more anti-cancer agents has been investigated, with the first, and the only, new clinical trial started in 2009. New clinical trials directed to such combinations have increased dramatically since then with 467 new trials registered in 2017 (C. Schmidt, Nature, Vol 552, 21/28 Dec. 2017). While the combination therapy of nivolumab and ipilimumab to treat melanoma, and the combination therapy of pemborlizumab with chemotherapy to treat non-small cell lung cancer were approved by the FDA in 2015 and 2017, respectively, there is a continued need of finding optimal therapies that combine a PD-1 axis binding antagonist with one or more other anti-cancer agents, for treating, stabilizing, preventing, and/or delaying development of various cancers.

### SUMMARY

Each of the embodiments described below can be combined with any other embodiment described herein not inconsistent with the embodiment with which it is combined. Furthermore, each of the embodiments described herein envisions within its scope pharmaceutically acceptable salts of the compounds described herein. Accordingly, the phrase “or a pharmaceutically acceptable salt thereof” is implicit in the description of all compounds described herein. Embodiments within an aspect as described below can be combined with any other embodiments not inconsistent within the same aspect.

In one embodiment, provided herein is a method for treating a subject having cancer, by administering to the subject:

(i) an amount of a PD-1 axis binding antagonist;

(ii) an amount of a CD-122-biased cytokine agonist, and

(iii) an amount of an anti-androgen, or a pharmaceutically acceptable salt thereof,

wherein the amounts together are effective in treating cancer.

In one aspect of the embodiment and in combination with any other aspects not inconsistent, the subject is a mammal. In some embodiments, the subject is a human.

In another aspect of the embodiment, and in combination with any other aspects not inconsistent, the cancer is prostate cancer. In some embodiments, the prostate cancer is metastatic. In some embodiments, the cancer is prostate cancer, which prostate cancer is castration resistant prostate cancer. In some embodiments, the cancer is prostate cancer, which prostate cancer is metastatic castration resistant prostate cancer (mCRPC).

In another aspect of the embodiment, and in combination with any other aspects not inconsistent, the anti-androgen is abiraterone or a pharmaceutically acceptable salt thereof, preferably abiraterone acetate. In another aspect of the embodiment, and in combination with any other aspects not inconsistent, the anti-androgen, or a pharmaceutically acceptable salt thereof, is an androgen receptor inhibitor, or a pharmaceutically acceptable salt thereof.

In another aspect of the embodiment, and in combination with any other aspects not inconsistent, the anti-androgen is an androgen receptor inhibitor. In some embodiments, the androgen receptor inhibitor is selected from the group consisting of enzalutamide, N-desmethyl enzalutamide, darolutamide, and apalutamide, or a pharmaceutically acceptable salt thereof.

In another aspect of the embodiment, and in combination with any other aspects not inconsistent, the anti-androgen is enzalutamide, or a pharmaceutically acceptable salt thereof. In some embodiments, the anti-androgen is enzalutamide.

In another aspect of the embodiment, and in combination with any other aspects not inconsistent, the anti-androgen is apalutamide, or a pharmaceutically acceptable salt thereof. In some embodiments, the anti-androgen is apalutamide.

In another aspect of the embodiment, and in combination with any other aspects not inconsistent, the CD-122 biased cytokine agonist is a long acting, IL-2Rβ-selective agonist composition comprising compounds of Formula (I),

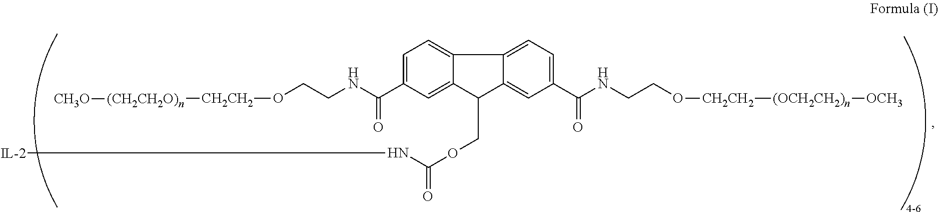

wherein IL-2 is an interleukin-2, “—NH-IL-2” represents an amino group of the interleukin-2, and each integer (n) has a value from about 3-4000, or from about 200-300, or pharmaceutically acceptable salts thereof, (referred to herein as (2,7-(bis-methoxyPEG-carboxyamide)(9H-fluorene-9-yl)methyl N-carbamate)₄₋₆interleukin-2 or “RSLAIL-2”). In some embodiments, the RSLAIL-2 composition contains no more than about 10 percent (molar) of compounds encompassed by the following formula:

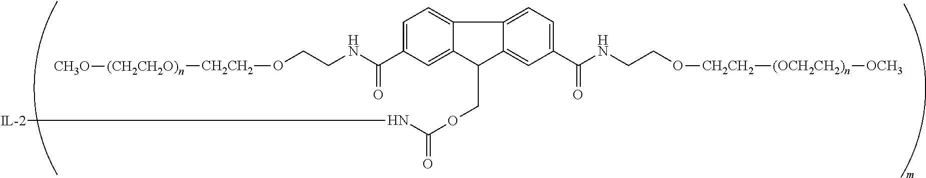

wherein (m) is an integer selected from the group consisting of 1, 2, 3, 7 and >7, or pharmaceutically acceptable salts thereof, and each integer (n) has a value from about 200-300. In some embodiments, for the RSLAIL-2, each of the “m” branched polyethylene glycol moieties of Formula (I) has a weight average molecular weight of about 20,000 daltons.

In another aspect of the embodiment, and in combination with any other aspects not inconsistent, the CD122-biased cytokine agonist is bempegaldesleukin. In some embodiments, bempegaldesleukin is administered as an intravenous (IV) dose in an amount of about 0.003 mg/kg to about 0.006 mg/kg Q2W.

In another aspect of the embodiment, and in combination with any other aspects not inconsistent, the PD-1 axis binding antagonist is selected from the group consisting atezolizumab (available from Genentech as TECENTRIQ®), avelumab (available from Merck KGaA and Pfizer as BAVENCIO®), durvalumab (available from AstraZeneca as nivolumab (available from Bristol-Myers Squibb as OPDIVO®), pembrolizumab (available from Merck as KEYTRUDA®), or tislelizumab (BeiGene BGB-A317).

In another aspect of the embodiment, and in combination with any other aspects not inconsistent, the PD-1 axis binding antagonist is avelumab. In some embodiments, avelumab is administered as an intravenous (IV) dose of about 10 mg/kg Q2W (one dose every two weeks). In some embodiments, avelumab is administered as an IV dose of about 800 mg Q2W.

In another aspect of the embodiment, and in combination with any other aspects not inconsistent, the combination of the PD-1 axis binding antagonist with a CD-122-biased cytokine agonist and an anti-androgen may be administered concurrently or sequentially, and in any order, and via the same and/or different routes of administration.

In some further embodiments of the method, the cancer is selected from, for example, the group consisting of head and neck cancer (including metastatic and recurring), breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, maxillary sinus cancer, bladder cancer, esophageal cancer, Hodgkin's disease and adrenocortical cancer.

In another aspect of the embodiment, and in combination with any other aspects not inconsistent, the cancer is a solid tumor. In some embodiments, the cancer is prostate cancer. In some embodiments, the cancer is prostate cancer which prostate cancer is high risk prostate cancer. In some embodiments, the cancer is prostate cancer which prostate cancer is locally advanced prostate cancer.

In some embodiments, the cancer is high risk locally advanced prostate cancer. In some embodiments, the cancer is prostate cancer which prostate cancer is castration-sensitive prostate cancer. Castration sensitive prostate cancer is also known as hormone sensitive prostate cancer. Hormone sensitive prostate cancer is usually characterised by histologically or cytologically confirmed adenocarcinoma of the prostate which is still responsive to androgen deprivation therapy. In some embodiments, the cancer is prostate cancer and the prostate cancer is non-metastatic castration sensitive prostate cancer. In some embodiments, the cancer is prostate cancer which prostate cancer is metastatic castration sensitive prostate cancer. In some embodiments, the cancer is prostate cancer, which prostate cancer is castration-resistant prostate cancer. Castration resistant prostate cancer is also know as hormone-refractory prostate cancer or androgen-independent prostate cancer. Castration resistant prostate cancer is usually characterised by histologically or cytologically confirmed adenocarcinoma of the prostate which is castration resistant (for example defined as 2 or more consecutive rises of PSA, ≥1 week between each assessment, optionally resulting in 2 or more 50% or greater increases over the nadir, with PSA level ≥2 ng/mL), in a setting of castrate levels of testosterone (for example ≤1.7 nmol/L level of testosterone or ≤50 ng/dL level of testosterone), which castrate levels of testosterone are achieved by androgen deprivation therapy and/or post orchiectomy. In some embodiments, the cancer is prostate cancer, which prostate cancer is non-metastatic castration-resistant prostate cancer.

In another aspect of the embodiment, and in combination with any other aspects not inconsistent, the cancer is prostate cancer, which prostate cancer is metastatic castration-resistant prostate cancer. In some embodiments, the subject having cancer has progressed on 1 line of abiraterone acetate/prednisone anti-androgen therapy for treatment of mCRPC. In some embodiments, the subject having cancer has had bilateral orchiectomy (surgical castration). In some embodiments, the subject having cancer is being treated with androgen deprivation therapy, for example with a gonadotropin releasing hormone (GnRH) agonist/antagonist (medical castration). In some embodiments, the subject has not received any prior chemotherapy for mCRPC, wherein prior treatment with radium 223 is allowed and it does not count for a line of prior chemotherapy. In some embodiments, the subject having cancer has received no prior treatment with enzalutamide. In some embodiments, the subject having cancer has received no prior treatment with apalutamide. In some embodiments, the subject having cancer has received no prior treatment with darolutamide. In some embodiments, the subject having cancer has progressed on 1 line of abiraterone acetate/prednisone anti-androgen therapy for treatment of mCRPC and has had bilateral orchiectomy (surgical castration). In some embodiments, the subject having cancer has progressed on 1 line of abiraterone acetate/prednisone anti-androgen therapy for treatment of mCRPC and is being treated with androgen deprivation therapy, for example with a gonadotropin releasing hormone (GnRH) agonist/antagonist (medical castration). In some embodiments, the subject having cancer has progressed on 1 line of abiraterone acetate/prednisone anti-androgen therapy for treatment of mCRPC, has had bilateral orchiectomy (surgical castration), and has not received any prior chemotherapy for mCRPC, wherein prior treatment with radium 223 is allowed and it does not count for a line of prior chemotherapy. In some embodiments, the subject having cancer has progressed on 1 line of abiraterone acetate/prednisone anti-androgen therapy for treatment of mCRPC, is being treated with androgen deprivation therapy, for example with a gonadotropin releasing hormone (GnRH) agonist/antagonist (medical castration) and has not received any prior chemotherapy for mCRPC, wherein prior treatment with radium 223 is allowed and it does not count for a line of prior chemotherapy. In some embodiments, the subject having cancer has progressed on 1 line of abiraterone acetate/prednisone anti-androgen therapy for treatment of mCRPC, has had bilateral orchiectomy (surgical castration), has not received any prior chemotherapy for mCRPC, wherein prior treatment with radium 223 is allowed and it does not count for a line of prior chemotherapy, and has received no prior treatment with enzalutamide, apalutamide or darolutamide. In some embodiments, the subject having cancer has progressed on 1 line of abiraterone acetate/prednisone anti-androgen therapy for treatment of mCRPC, is being treated with androgen deprivation therapy, for example with a gonadotropin releasing hormone (GnRH) agonist/antagonist (medical castration), has not received any prior chemotherapy for mCRPC, wherein prior treatment with radium 223 is allowed and it does not count for a line of prior chemotherapy, and has received no prior treatment with enzalutamide, apalutamide or darolutamide.

In another aspect of the embodiment, and in combination with any other aspects not inconsistent, the PD-1 axis binding antagonist is administered to the subject prior to administering the CD-122-biased cytokine agonist (such as RSLAIL-2). In some particular embodiments, the PD-1 axis binding antagonist and the CD-122-biased cytokine agonist (such as RSLAIL-2) are both administered on day 1 of treatment. In yet some additional embodiments, the PD-1 axis binding antagonist is administered on day 1 of treatment and the CD-122-biased cytokine agonist (such as RSLAIL-2) is administered on a day greater than 5 days following administration of the PD-1 axis binding antagonist (e.g., on day 6, 7, 8, 9, 10, 11, 12, 13, 14, 15, or greater, of treatment).

In another aspect of the embodiment, and in combination with any other aspects not inconsistent, the CD-122-biased cytokine agonist is RSLAIL-2. In some embodiments, the RSLAIL-2 is bempegaldesleukin. In some embodiments, the amount of bempegaldesleukin is an IV dose of about 0.0001 mg/kg to about 0.1 mg/kg body weight Q2W.

In another aspect of the embodiment, and in combination with any other aspects not inconsistent, the PD-1 axis binding antagonist is avelumab and is administered in an IV dose of 800 mg Q2W, the anti-androgen is enzalutamide and is administered at an oral dose of 160 mg QD, 120 mg QD or 80 mg QD, the CD-122 biased cytokine agonist is bempegaldesleukin and is administered as an IV dose of about 0.003 mg/kg to 0.006 mg/kg Q2W, and the cancer is mCRPC. In another aspect of the embodiment, and in combination with any other aspects not inconsistent, the PD-1 axis binding antagonist is avelumab and is administered in an IV dose of 800 mg Q2W, the anti-androgen is enzalutamide and is administered at an oral dose of 160 mg QD, the CD-122 biased cytokine agonist is bempegaldesleukin and is administered as an IV dose of about 0.003 mg/kg, and the cancer is mCRPC. In another aspect of the embodiment, and in combination with any other aspects not inconsistent, the PD-1 axis binding antagonist is avelumab and is administered in an IV dose of 800 mg Q2W, the anti-androgen is enzalutamide and is administered at an oral dose of 120 mg QD, the CD-122 biased cytokine agonist is bempegaldesleukin and is administered as an IV dose of about 0.003 mg/kg Q2W, and the cancer is mCRPC. In another aspect of the embodiment, and in combination with any other aspects not inconsistent, the PD-1 axis binding antagonist is avelumab and is administered in an IV dose of 800 mg Q2W, the anti-androgen is enzalutamide and is administered at an oral dose of 80 mg QD, the CD-122 biased cytokine agonist is bempegaldesleukin and is administered as an IV dose of about 0.003 mg/kg Q2W, and the cancer is mCRPC. In another aspect of the embodiment, and in combination with any other aspects not inconsistent, the PD-1 axis binding antagonist is avelumab and is administered in an IV dose of 800 mg Q2W, the anti-androgen is enzalutamide and is administered at an oral dose of 160 mg QD, the CD-122 biased cytokine agonist is bempegaldesleukin and is administered as an IV dose of about 0.006 mg/kg Q2W, and the cancer is mCRPC. In another aspect of the embodiment, and in combination with any other aspects not inconsistent, the PD-1 axis binding antagonist is avelumab and is administered in an IV dose of 800 mg Q2W, the anti-androgen is enzalutamide and is administered in an oral dose of 120 mg QD, the CD-122 biased cytokine agonist is bempegaldesleukin and is administered as an IV dose of about 0.006 mg/kg Q2W, and the cancer is mCRPC. In another aspect of the embodiment, and in combination with any other aspects not inconsistent, the PD-1 axis binding antagonist is avelumab and is administered in an IV dose of 800 mg Q2W, the anti-androgen is enzalutamide and is administered at an oral dose of 80 mg QD, the CD-122 biased cytokine agonist is bempegaldesleukin and is administered as an IV dose of about 0.006 mg/kg Q2W, and the cancer is mCRPC. In some embodiments, avelumab and bempegaldesleukin are administered on the same day. In some embodiments, bempegaldesleukin is administered on the same day of, and prior to, the administration of avelumab. In some embodiments, bempegaldesleukin is administered on the same day of, and after, the administration of avelumab. in some embodiments, prior to the first, first two, first three or first four administrations of avelumab, the patient is premedicated with an antihistamine and or acetaminophen. In some embodiments, enzalutamide is administered before avelumab and bempegaldesleukin. In some embodiments, enzalutamide is administered after avelumab and bempegaldesleukin.

In some embodiments of the method, each of the PD-1 axis binding antagonist and the CD-122-biased cytokine agonist, and the anti-androgen, or a pharmaceutically acceptable salt thereof, are administered as separate compositions.

In yet additional embodiments, the cancer comprises a cancerous tumor and the method is effective to reduce the size of the cancerous tumor when compared to the size of the tumor prior to the administering. Or in some more particular embodiments, the cancer comprises a cancerous tumor and the method is effective to reduce the size of the cancerous tumor by at least about 30% (partial response), or by at least about 40%, or by at least about 50%, or by at least about 60%, or by at least about 70%, or at least about 80%, or at least about 90%, or to result in complete tumor regression, when compared to the size of the tumor prior to the administering. In yet some further embodiments of the method, the cancer comprises a cancerous tumor and the method is effective to result in complete tumor regression.

In some embodiments relating to any one or more of the foregoing aspects, when treating a solid cancerous tumor, the method is effective to result in a reduction in solid tumor size of at least about 25% when evaluated after 1 cycle of treatment.

In some embodiments relating to any one or more of the foregoing aspects, when treating prostate cancer, the method is effective to result in a decrease in prostate specific antigen (PSA) from baseline. In some embodiments, when treating prostate cancer, the method is effective to result in a decrease in PSA of greater than or equal to 50% from baseline. In some embodiments, when treating prostate cancer, the method is effective to result in a decrease in PSA of greater than or equal to 50% from baseline confirmed by a second consecutive assessment at least 3 weeks after baseline assessment. In some embodiments, when treating prostate cancer, the method is effective to result in no evidence of confirmed bone disease progression from baseline on repeat bone scan at least 6 weeks after baseline assessment. In some embodiments, when treating prostate cancer, the method is effective to result in a decrease in circulating tumor cell (CTC) count from greater than or equal to 5 CTC per 7.5 mL of blood at baseline to less than 5 CTC per 7.5 mL of blood at assessment. In some embodiments, when treating prostate cancer, the method is effective to result in a decrease in circulating tumor cell (CTC) count from greater than or equal to 1 CTC per 7.5 mL of blood at baseline 0 CTC per 7.5 mL of blood at assessment.

In yet an additional embodiment, in connection with the treatment of patients suffering from castration-resistant prostate cancer, the method of treatment comprises administering to the individual a combination of a PD-1 axis binding antagonist with a CD-122-biased cytokine agonist, and an anti-androgen, or a pharmaceutically acceptable salt thereof.

Additional aspects and embodiments are set forth in the following description and claims.

### DETAILED DESCRIPTION OF THE INVENTION

The instant application relates to cancer therapy. Certain embodiments relate to the treatment of an individual having cancer by administering to the individual a combination of a PD-1 axis binding antagonist with a CD-122-biased cytokine agonist, and an anti-androgen, or a pharmaceutically acceptable salt thereof.

### Definitions

In describing and claiming certain features of this disclosure, the following terminology will be used in accordance with the definitions described below unless indicated otherwise.

As used in this specification, the singular forms “a,” “an,” and “the” include plural referents unless the context clearly dictates otherwise.

Similarly, “about” when used to modify a numerically defined parameter (e.g., the dose of a PD-1 axis binding antagonist, or the length of treatment time with a combination therapy described herein) means that the parameter may vary by as much as 10% below or above the stated numerical value for that parameter. For example, a dose of about 5 mg/kg may vary between 4.5 mg/kg and 5.5 mg/kg. “About” when used at the beginning of a listing of parameters is meant to modify each parameter. For example, about 0.5 mg, 0.75 mg or 1.0 mg means about 0.5 mg, about 0.75 mg or about 1.0 mg. Likewise, about 5% or more, 10% or more, 15% or more, 20% or more, and 25% or more means about 5% or more, about 10% or more, about 15% or more, about 20% or more, and about 25% or more.

“Administering” refers to the delivery of a therapeutic agent to a subject, using any of the various methods and delivery systems known to those skilled in the art. Exemplary routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

A “chemotherapeutic agent” is a chemical compound useful in the treatment of cancer. Examples of chemotherapeutic agents include alkylating agents such as thiotepa and cyclophosphamide (CYTOXAN®); alkyl sulfonates such as busulfan, improsulfan, and piposulfan; aziridines such as. benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB 1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma II and calicheamicin omegal I (see, e.g., Nicolaou et al, Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HC1 liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-it inhibitors; anti-adrenals such as aminoglutethimide, mitotane, trilostane; folic acid replenisher such as frolinic acid; aceglatone; aldophosphamide glycoside; am inolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDIS1NE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylomithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

A “chemotherapy” as used herein, refers to a chemotherapeutic agent, as defined above, or a combination of two, three or four chemotherapeutic agents, for the treatment of cancer. When a chemotherapy consists more than one chemotherapeutic agents, the chemotherapeutic agents can be administered to the patient on the same day or on different days in the same treatment cycle.

It is understood that wherever aspects are described herein with the language “comprising,” otherwise analogous aspects described in terms of “consisting of” and/or “consisting essentially of” are also provided.

As used herein the term “anti-androgen”, and “anti-androgens” shall be taken to mean compounds which prevent androgens, for example testosterone and dihydrotestosterone (DHT) and the like, from mediating their biological effects in the body. Anti-androgens may act by one or more of the following hormonal mechanisms of action such as blocking and/or inhibiting and/or modulating the androgen receptor (AR); inhibiting androgen production; suppressing androgen production; degrading the AR, inhibiting nuclear translocation, inhibiting binding of the AR to nuclear DNA, and the like. Anti-androgens include, but are not limited to, steroidal androgen receptor inhibitors (for example, cyproterone acetate, spironolactone, megestrol acetate, chlormadinone acetate, oxendolone, and osaterone acetate), non-steroidal androgen receptor inhibitors (for example, enzalutamide, bicalutamide, nilutamide, flutamide, topilutamide, apalutamide), androgen synthesis inhibitors, androgen receptor degraders and the like.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also antigen binding fragments thereof (such as Fab, Fab′, F(ab′)2, Fv), single chain (scFv) and domain antibodies (including, for example, shark and camelid antibodies), and fusion proteins comprising an antibody, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

“Branched,” in reference to the geometry or overall structure of a polymer, refers to a polymer having two or more polymer “arms” or “chains” extending from a branch point or central structural feature.

A “cancer” refers a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, hodgkin's lymphoma, non-hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer. Another particular example of cancer includes renal cell carcinoma.

The term “CD-122-biased cytokine agonist” (also referred to as an interleukin-2 receptor beta (ILRβ), selective agonist) as used herein, refers to an agonist that has a greater affinity for binding to IL-2Rβ than to IL-2Rαβ. By way of example, it is possible to measure binding affinities relative to IL-2 as a standard using surface plasmon resonance (using, e.g., a system such as BIACORE™ T100). Generally, a CD122-biased agonist will possess an in vitro binding affinity for IL-2Rβ that is at least 5 times greater (more preferably at least 10 times greater) than the binding affinity for IL-2Rαβ in the same in vitro model. In this regard, bempegaldesleukin exhibits about a 60-fold decrease in affinity to IL-2Rαβ relative to IL-2, but only about a 5-fold decrease in affinity IL-2Rβ relative to IL-2.

As used herein, an “effective dosage” or “effective amount” of drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various diseases or conditions (such as for example cancer), decreasing the dose of other medications required to treat the disease, enhancing the effect of another medication, and/or delaying the progression of the disease. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

The term “immunotherapy” refers to the treatment of a subject by a method comprising inducing, enhancing, suppressing, or otherwise modifying an immune response.

The term “patient,” or “subject” as used herein refers to a living organism suffering from or prone to a condition that can be prevented or treated by administration of a compound or composition or combination as provided herein, such as a cancer, and includes both humans and animals. Subjects include, but are not limited to, mammals (e.g., murines, simians, equines, bovines, porcines, canines, felines, and the like), and preferably are human

The term “PD-1 axis binding antagonist” as used herein refers to a molecule that inhibits the interaction of a PD-1 axis binding partner with either one or more of its binding partner, so as to remove T-cell dysfunction resulting from signaling on the PD-1 signaling axis, with a result being to restore or enhance T-cell function. As used herein, a PD-1 axis binding antagonist includes a PD-1 binding antagonist, a PD-L1 binding antagonist and a PD-L2 binding antagonist.

The term “PD-1 binding antagonist” as used herein refers to a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less non-dysfunctional. In some embodiments, the PD-1 binding antagonist is an anti-PD-1 antibody. In a specific aspect, a PD-1 binding antagonist is nivolumab. In another specific aspect, a PD-1 binding antagonist is pembrolizumab. In another specific aspect, a PD-1 binding antagonist is pidilizumab.

The term “PD-L1 binding antagonist” as used herein refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding antagonist reduces the negative co-stimulatory signal mediated by or through cell surface proteins expressed on T lymphocytes mediated signaling through PD-L1 so as render a dysfunctional T-cell less non-dysfunctional. In some embodiments, a PD-L1 binding antagonist is an anti-PD-L1 antibody. In a specific aspect, an anti-PD-L1 antibody is avelumab. In another specific aspect, an anti-PD-L1 antibody is atezolizumab. In another specific aspect, an anti-PD-L1 antibody is durvalumab. In another specific aspect, an anti-PD-L1 antibody is BMS-936559 (MDX-1105).

As used herein, an anti-human PD-L1 antibody refers to an antibody that specifically binds to mature human PD-L1. A mature human PD-L1 molecule consists of amino acids 19-290 of the following sequence: SEQ ID NO:1:

MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET.

The term “PD-L2 binding antagonists” as used herein refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less non-dysfunctional. In some embodiments, a PD-L2 binding antagonist is a PD-L2 immunoadhesin.

“PEG” or “polyethylene glycol,” as used herein, is meant to encompass any water-soluble poly(ethylene oxide). Unless otherwise indicated, a “PEG polymer” or a polyethylene glycol is one in which substantially all (preferably all) monomeric subunits are ethylene oxide subunits, though, the polymer may contain distinct end capping moieties or functional groups, e.g., for conjugation. PEG polymers for use in the present invention will comprise one of the two following structures: “—(CH₂CH₂O)ₙ—” or “—(CH₂CH₂O)ₙ₋₁CH₂CH₂—,” depending upon whether or not the terminal oxygen(s) has been displaced, e.g., during a synthetic transformation. As stated above, for the PEG polymers, the variable (n) can range from about 3 to 4000, but may also fall within a subset of such range, and the terminal groups and architecture of the overall PEG can vary.

“Pharmaceutically acceptable excipient” or “pharmaceutically acceptable carrier” refers to a component that may be included in the compositions described herein and causes no significant adverse toxicological effects to a subject.

The terms “protein”, “polypeptide” and “peptide” are used interchangeably herein and refer to any peptide-linked chain of amino acids, regardless of length co-translational or post-translational modification.

A covalent “releasable” linkage, for example, in the context of a polyethylene glycol that is covalently attached to an active moiety such as interleukin-2, is one that releases under physiological conditions by any suitable release mechanism to thereby release or detach a polyethylene glycol polymer from the active moiety.

“Substantially” or “essentially” means nearly totally or completely, for instance, 95% or greater of a given quantity.

The term “substantially homologous” or “substantially identical” means that a particular subject sequence, for example, a mutant sequence, varies from a reference sequence by one or more substitutions, deletions, or additions, the net effect of which does not result in an adverse functional dissimilarity between the reference and subject sequences. For purposes herein, a sequence having greater than 95 percent homology (identity), equivalent biological activity (although not necessarily equivalent strength of biological activity), and equivalent expression characteristics to a given sequence is considered to be substantially homologous (identical). For purposes of determining homology, truncation of the mature sequence should be disregarded.

The terms “synergy” or “synergistic” are used to mean that the result of the combination of two or more compounds, components or targeted agents is greater than the sum of each agent together. The terms “synergy” or “synergistic” also means that there is an improvement in the disease condition or disorder being treated, over the use of the two or more compounds, components or targeted agents while each compound, component or targeted agent is used individually. This improvement in the disease condition or disorder being treated is a “synergistic effect”. A “synergistic amount” is an amount of the combination of the two compounds, components or targeted agents that results in a synergistic effect, as “synergistic” is defined herein. Determining a synergistic interaction between one or two components, the optimum range for the effect and absolute dose ranges of each component for the effect may be definitively measured by administration of the components over different w/w (weight per weight) ratio ranges and doses to patients in need of treatment. However, the observation of synergy in in vitro models or in vivo models can be predictive of the effect in humans and other species and in vitro models or in vivo models exist, as described herein, to measure a synergistic effect and the results of such studies can also be used to predict effective dose and plasma concentration ratio ranges and the absolute doses and plasma concentrations required in humans and other species by the application of pharmacokinetic/pharmacodynamic methods.

The term “treat” or “treating” a cancer as used herein means to administer a combination therapy according to the present invention to a subject having cancer, or diagnosed with cancer, to achieve at least one positive therapeutic effect, such as, for example, reduced number of cancer cells, reduced tumor size, reduced rate of cancer cell infiltration into peripheral organs, or reduced rate of tumor metastases or tumor growth, reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. For the purposes of this invention, beneficial or desired clinical results include, but are not limited to, one or more of the following: reducing the proliferation of (or destroying) neoplastic or cancerous cell; inhibiting metastasis or neoplastic cells; shrinking or decreasing the size of tumor; remission of the cancer; decreasing symptoms resulting from the cancer; increasing the quality of life of those suffering from the cancer; decreasing the dose of other medications required to treat the cancer; delaying the progression the cancer; curing the cancer; overcoming one or more resistance mechanisms of the cancer; and/or prolonging survival of patients the cancer. Positive therapeutic effects in cancer can be measured in a number of ways (see, for example, W. A. Weber, J. Nucl. Med. 50:1S-10S (200)). In some embodiments, the treatment achieved by a combination of the invention is any of the partial response (PR), complete response (CR), overall response (OR), objective response rate (ORR), progression free survival (PFS), radiographic PFS, disease free survival (DFS) and overall survival (OS). PFS, also referred to as “Time to Tumor Progression” indicates the length of time during and after treatment that the cancer does not grow, and includes the amount of time patients have experience a CR or PR, as well as the amount of time patients have experience stable disease (SD). DFS refers to the length of time during and after treatment that the patient remains free of disease. OS refers to a prolongation in life expectancy as compared to naïve or untreated subjects or patients. In some embodiments, response to a combination of the invention is any of PR, CR, PFS, DFS, ORR, OR or OS. Response to a combination of the invention, including duration of soft tissue response, is assessed using Response Evaluation Criteria in Solid Tumors version 1.1 (RECIST 1.1) response criteria. In some embodiments, the treatment achieved by a combination of the invention is measured by the time to PSA progression, the time to initiation of cytotoxic chemotherapy and the proportion of patients with PSA response greater than or equal to 50%. The treatment regimen for a combination of the invention that is effective to treat a cancer patient may vary according to factors such as the disease state, age, and weight of the patient, and the ability of the therapy to elicit an anti-cancer response in the subject. While an embodiment of any of the aspects of the invention may not be effective in achieving a positive therapeutic effect in every subject, it should do so in a statistically significant number of subjects as determined by any statistical test known in the art such as, but not limited to, the Cox log-rank test, the Cochran-Mantel-Haenszel log-rank test, the Student's t-test, the chi2-test, the U-test according to Mann and Whitney, the Kruskal-Wallis test (H-test), Jonckheere-Terpstrat-test and the Wilcon on-test. The term “treatment” also encompasses in vitro and ex vivo treatment, e.g., of a cell, by a reagent, diagnostic, binding compound, or by another cell.

Molecular weight in the context of a water-soluble polymer, such as PEG, can be expressed as either a number average molecular weight or a weight average molecular weight. Unless otherwise indicated, all references to molecular weight herein refer to the weight average molecular weight. Both molecular weight determinations, number average and weight average, can be measured using gel permeation chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight or the use of light scattering techniques, ultracentrifugation, or viscometry to determine weight average molecular weight. PEG polymers are typically polydisperse (i.e., number average molecular weight and weight average molecular weight of the polymers are not equal), possessing low polydispersity values of preferably less than about 1.2, more preferably less than about 1.15, still more preferably less than about 1.10, yet still more preferably less than about 1.05, and most preferably less than about 1.03.

### Methods, Uses and Medicaments

Provided herein is a combination method based upon administration of a combination of a PD-1 axis binding antagonist with a CD-122-biased cytokine agonist, and an anti-androgen, or a pharmaceutically acceptable salt thereof.

PD-1 Axis Binding Antagonist

The combinations and methods provided herein comprise at least one PD-1 axis binding antagonist. Administration of the PD-1 axis binding antagonist is effective to, for example, enhance T cell cytolytic activity.

Illustrative PD-1 axis binding antagonists include, but are not limited to, for example: avelumab (BAVENCIO®, MSB0010718C, Merck KGaA), atezolizumab (TECENTRIQ®, MPDL3280A, Roche Holding AG), durvalumab (IMFINZI®, AstraZeneca PLC), nivolumab (OPDIVO®, ONO-4538, BMS-936558, MDX1106, Bristol-Myers Squibb Company), pembrolizumab (KEYTRUDA®, MK-3475, lambrolizumab, Merck & Co., Inc.), BCD100 (BIOCAD Biopharmaceutical Company), BGB-A317 (BeiGene Ltd./Celgene Corporation), CBT-501 (CBT Pharmaceuticals), CBT-502 (CBT Pharmaceuticals), GLS-010 (Harbin Gloria Pharmaceuticals Co., Ltd.), 161308 (Innovent Biologics, Inc.), WBP3155 (CStone Pharmaceuticals Co., Ltd.), AMP-224 (GlaxoSmithKline plc), BI 754091 (Boehringer Ingelheim GmbH), BMS-936559 (Bristol-Myers Squibb Company), CA-170 (Aurigene Discovery Technologies), FAZ053 (Novartis AG), PDR001 (Novartis AG), LY3300054 (Eli Lilly & Company), M7824 (Merck KGaA), MEDI0680 (AstraZeneca PLC), PDR001 (Novartis AG), PF-06801591 (aka RN888) (Pfizer Inc.), described as mAb7 in International Patent Publication No. WO2016/092419, the disclosure of which is hereby incorporated by reference in its entirety, REGN2810 (Regeneron Pharmaceuticals, Inc.), SHR-1210 (Incyte Corporation), TSR-042 (Tesaro, Inc.), AGEN2034 (Agenus Inc.), CX-072 (CytomX Therapeutics, Inc.), JNJ-63723283 (Johnson & Johnson), MGD013 (MacroGenics, Inc.), AN-2005 (Adlai Nortye), ANA011 (AnaptysBio, Inc.), ANB011 (AnaptysBio, Inc.), AUNP-12 (Pierre Fabre Medicament S.A.), BBI-801 (Sumitomo Dainippon Pharma Co., Ltd.), BION-004 (Aduro Biotech), CA-327 (Aurigene Discovery Technologies), CK-301 (Fortress Biotech, Inc.), ENUM 244C8 (Enumeral Biomedical Holdings, Inc.), FPT155 (Five Prime Therapeutics, Inc.), FS118 (F-star Alpha Ltd.), hAb21 (Stainwei Biotech, Inc.), J43 (Transgene S.A.), JTX-4014 (Jounce Therapeutics, Inc.), KD033 (Kadmon Holdings, Inc.), KY-1003 (Kymab Ltd.), MCLA-134 (Merus B.V.), MCLA-145 (Merus B.V.), PRS-332 (Pieris AG), SHR-1316 (Atridia Pty Ltd.), STI-A1010 (Sorrento Therapeutics, Inc.), STI-A1014 (Sorrento Therapeutics, Inc.), STI-A1110 (Les Laboratoires Servier), and XmAb20717 (Xencor, Inc.).

BGB-A317 (tislelizumab), under development by BeiGene Ltd., is a humanized IgG4, monoclonal antibody having an engineered Fc region (i.e., where the ability to bind Fc gamma receptor I has been specifically removed). BGB-A317 binds to PD-1 and inhibits the binding of PD-1 to PD-L1 and PD-L2.

Avelumab (BAVENCIO®, MSB0010718C) is disclosed as A09-246-2, in International Patent Publication No. WO2013/079174, the disclosure of which is hereby incorporated by reference in its entirety.

In one or more embodiments, the PD-1 axis binding antagonist is selected from avelumab, atezolizumab, durvalumab, nivolumab, pembrolizumab, and BGB-A317.

In accordance with the methods described herein, an effective amount of a PD-1 axis binding antagonist may be administered. One of ordinary skill in the art can determine how much of the PD-1 axis binding antagonist is sufficient to provide clinically relevant inhibition. For example, one of ordinary skill in the art can refer to the literature and/or administer a series of increasing amounts of the PD-1/PD-L1 axis inhibitor to determine which amount or amounts provide clinically relevant activity.

In some of the embodiments, the PD-1 axis binding antagonist is administered in the amount of from about 1 mg/kg to about 1000 mg/kg; from about 2 mg/kg to about 900 mg/kg; from about 3 mg/kg to about 800 mg/kg; from about 4 mg/kg to about 700 mg/kg; from about 5 mg/kg to about 600 mg/kg; from about 6 mg/kg to about 550 mg/kg; from about 7 mg/kg to about 500 mg/kg; from about 8 mg/kg to about 450 mg/kg; from about 9 mg/kg to about 400 mg/kg; from about 5 mg/kg to about 200 mg/kg; from about 2 mg/kg to about 150 mg/kg; from about 5 mg/kg to about 100 mg/kg; from about 10 mg/kg to about 100 mg/kg; and from about 10 mg/kg to about 60 mg/kg, in a weekly, biweekly, Q3W, Q4W, or Q6W, IV or subcutaneous dosing schedule. in some embodiments, the PD-1 axis binding antagonist is administered in the amount of about 5 mg/kg, about 10 mg/kg, about 15 mg/kg, about 400 mg to about 1000 mg, about 600 mg, about 700 mg, about 800 mg, about 900 mg, in a weekly, biweekly, Q3W, Q4W, or Q6W, IV or subcutaneous dosing schedule.

Long Acting, IL-2Rβ-Biased Agonist, RSLAIL-2

The combinations and methods described herein comprise a CD-122-biased cytokine agonist, such as the long acting, IL-2Rβ-biased agonist, RSLAIL-2 (encompassing pharmaceutically acceptable salt forms thereof), the preparation of which is described in Example 1 of U.S. Pat. No. 10,010,587. RSLAIL-2 exhibits about a 60-fold decrease in affinity to IL-2Rαβ relative to IL-2, but only about a 5-fold decrease in affinity IL-2Rβ relative to IL-2.

The releasable PEG comprised in RSLAIL-2 is based upon a 2,7,9-substituted fluorene as shown below, with poly(ethylene glycol) chains extending from the 2- and 7-positions on the fluorene ring via amide linkages (fluorene-C(O)—NH˜), and having releasable covalent attachment to IL-2 via attachment to a carbamate nitrogen atom attached via a methylene group (—CH₂—) to the 9-position of the fluorene ring. In this regard, RSLAIL-2 is a composition comprising compounds encompassed by the following formula:

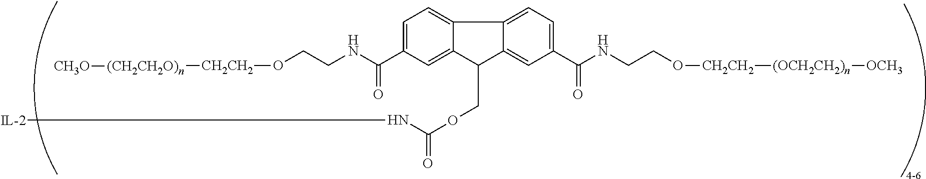

wherein IL-2 is an interleukin-2, and pharmaceutically acceptable salts thereof, where each “n” is an integer from about 3 to about 4000, or more preferably is an integer from about 200-300. In some preferred embodiments, each “n” is approximately the same. That is to say, the weight average molecular weight of each polyethylene glycol “arm” covalently attached to the fluorenyl core is about the same. In some preferred embodiments, the weight average molecular weight of each PEG arm is about 10,000 daltons, such that the weight average molecular weight of the overall branched polymer moiety is about 20,000 daltons. In one or more embodiments, the composition contains no more than 10% (based on a molar amount), and preferably no more than 5% (based on a molar amount), of compounds encompassed by the following formula

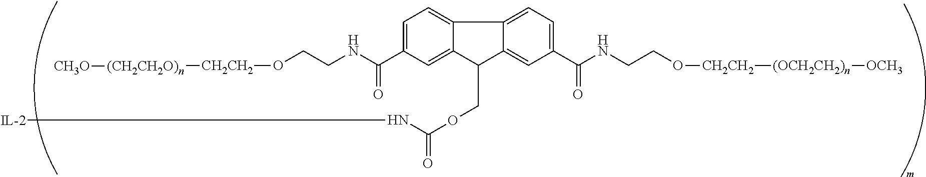

wherein IL-2 is an interleukin-2, and “m” (referring to the number of polyethylene glycol moieties attached to IL-2) is an integer selected from the group consisting of 1, 2, 3, 7 and >7, or pharmaceutically acceptable salts thereof.

In some preferred embodiments, RSLAIL-2 possesses on average about six of the branched fluorenyl-based polyethylene glycol moieties attached to IL-2.

In one or more embodiments, the long acting IL-2Rβ-biased agonist is encompassed by the following structure:

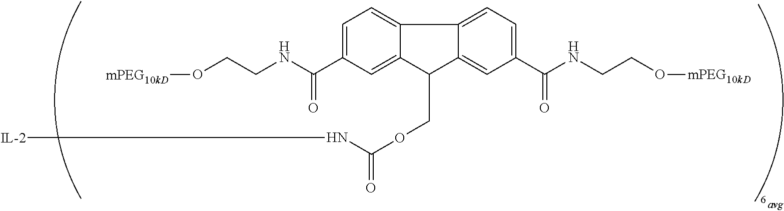

wherein IL-2 is recombinant human interleukin-2 (de-1-alanine, 125-serine), and each mPEG_{10kD} has a structure —CH₂CH₂(OCH₂CH₂)ₙOCH₃, where “n” has an approximate value of about 227 on average. The preparation of the foregoing is described, e.g., in WO 2018/132496 (Example 19), while Example 20 describes the molecule's receptor bias.

“Bempegaldesleukin”, as used herein refers to (2,7-(bis-methoxyPEG_{10kD}-carboxyamide)(9H-fluorene-9-yl)methyl N-carbamate)_{6avg} interleukin-2 (CAS No. 1939126-74-5), a CD-122 biased cytokine agonist in which recombinant human interleukin-2 (de-1-alanine, 125-serine), is N-substituted with an average of six [(2,7-bis{[methylpoly(oxyethylene)_{10kD}]carbamoyl}-9H-fluoren-9-yl)methoxy]carbonyl moieties at its amino residues.

Additional features of bempegaldesleukin are described in, e.g., Charych, D., et al., Clin Cancer Res, 2016; 22(3): 680-690, and Charych, D., et al., PLOS ONE, Jul. 5, 2017, p. 1-24.

To determine average degree of PEGylation for a composition such as described in Formula (I), typically the protein is quantified by a method such as an bicinchoninic acid (BCA) assay or by UV analysis, to determine moles of protein in the sample. The PEG moieties are then released by exposing the sample to conditions in which the PEG moieties are released, and the released PEG is then quantified (e.g., by BCA or UV) and correlated with moles protein to determine the average degree of PEGylation.

RSLAIL-2 can be considered to be an inactive prodrug, i.e., it is inactive upon administration, and by virtue of slow release of the polyethylene glycol moieties in vivo, provides active conjugated forms of interleukin-2 that are effective to achieve sustained concentrations at a tumor site.

Additional exemplary compositions of RSLAIL-2 comprise compounds in accordance with the above formulae wherein the overall branched polymer portion of the molecule has a weight average molecular weight in a range of from about 250 Daltons to about 90,000 Daltons. Additional suitable ranges include weight average molecular weights in a range selected from about 1,000 Daltons to about 60,000 Daltons, in a range of from about 5,000 Daltons to about 60,000 Daltons, in a range of about 10,000 Daltons to about 55,000 Daltons, in a range of from about 15,000 Daltons to about 50,000 Daltons, and in a range of from about 20,000 Daltons to about 50,000 Daltons.

Additional illustrative weight-average molecular weights for the polyethylene glycol polymer portion include about 200 Daltons, about 300 Daltons, about 400 Daltons, about 500 Daltons, about 600 Daltons, about 700 Daltons, about 750 Daltons, about 800 Daltons, about 900 Daltons, about 1,000 Daltons, about 1,500 Daltons, about 2,000 Daltons, about 2,200 Daltons, about 2,500 Daltons, about 3,000 Daltons, about 4,000 Daltons, about 4,400 Daltons, about 4,500 Daltons, about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, and about 75,000 Daltons. In some embodiments, the weight-average molecular weight of the branched polyethylene glycol polymer is about 20,000 daltons.

As described above, RSLAIL-2 may be in the form of a pharmaceutically-acceptable salt. Typically, such salts are formed by reaction with a pharmaceutically-acceptable acid or an acid equivalent. The term “pharmaceutically-acceptable salt” in this respect, will generally refer to the relatively non-toxic, inorganic and organic acid addition salts. These salts can be prepared in situ in the administration vehicle or the dosage form manufacturing process, or by separately reacting a long-acting interleukin-2 as described herein with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, oxylate, mesylate, glucoheptonate, lactobionate, and laurylsulphonate salts and the like. (See, for example, Berge et al. (1977) “Pharmaceutical Salts”, J. Pharm. Sci. 66:1-19). Thus, salts as described may be derived from inorganic acids such as hydrochloride, hydrobromic, sulfuric, sulfamic, phosphoric, nitric, and the like; or prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In reference to RSLAIL-2, the term “IL-2” as used herein, refers to a moiety having human IL-2 activity. The term, ‘residue’, in the context of residue of IL-2, when used, means the portion of the IL-2 molecule that remains following covalent attachment to a polymer such as a polyethylene glycol, at one or more covalent attachment sites, as shown in the formula above. It will be understood that when the unmodified IL-2 is attached to a polymer such as polyethylene glycol, the IL-2 is slightly altered due to the presence of one or more covalent bonds associated with linkage to the polymer(s). This slightly altered form of the IL-2 attached to another molecule is sometimes referred to a “residue” of the IL-2.

Proteins having an amino acid sequence corresponding to any one of SEQ ID NOs: 1 through 4 described in International Patent Publication No. WO 2012/065086 are exemplary IL-2 proteins. The term substantially homologous means that a particular subject sequence, for example, a mutant sequence, varies from a reference sequence by one or more substitutions, deletions, or additions, the net effect of which does not result in an adverse functional dissimilarity between the reference and subject sequences. For the purposes herein, sequences having greater than 95 percent homology, equivalent biological activity (although not necessarily equivalent strength of biological activity), and equivalent expression characteristics are considered substantially homologous. For purposes of determining homology, truncation of the mature sequence should be disregarded. The IL-2 may be naturally-occurring or may be recombinantly produced. In addition, the IL-2 can be derived from human sources, animal sources, and plant sources. Most preferably, the IL-2 is aldesleukin.

RSLAIL-2 is generally referred to as long-acting. For the purposes herein, the long acting nature of an IL-2Rβ biased agonist is typically determined using flow cytometry to measure STAT5 phosphorylation in lymphocytes at various time points after administration of the agonist to be evaluated in mice. As a reference, the signal is lost by around 24 hours with IL-2, but is sustained for a period greater than that for a long-acting IL-2Rβ-biased agonist. As an illustration, the signal is sustained over several days for RSLAIL-2.

In accordance with the method and compositions, described herein, RSLAIL-2 is provided in an IL-2Rβ-activating amount. One of ordinary skill in the art can determine how much RSLAIL-2 is sufficient to provide clinically relevant agonistic activity at IL-2Rβ. For example, one of ordinary skill in the art can refer to the literature and/or administer a series of increasing amounts of RSLAIL-2 and determine which amount or amounts provide clinically effective agonistic activity of IL-2Rβ. Alternatively, an activating amount of RSLAIL-2 can be determined using the in vivo STAT5 phosphorylation assay where an amount sufficient to induce STAT5 phosphorylation in greater than 10% of NK cells at peak is considered to be an activating amount.

In one or more instances, however, the IL-2Rβ-activating amount of RSLAIL-2 is an amount encompassed by one or more of the following ranges expressed in amount of protein: from about 0.01 to 100 mg/kg; from about 0.01 mg/kg to about 75 mg/kg; from about 0.02 mg/kg to about 60 mg/kg; from about 0.03 mg/kg to about 50 mg/kg; from about 0.05 mg/kg to about 40 mg/kg; from about 0.05 mg/kg to about 30 mg/kg; from about 0.05 mg/kg to about 25 mg/kg; from about 0.05 mg/kg to about 15 mg/kg; from about 0.05 mg/kg to about 10 mg/kg; from about 0.05 mg/kg to about 5 mg/kg; from about 0.05 mg/kg to about 1 mg/kg. In some embodiments, RSLAIL-2 is administered at a dose that is less than or equal to 0.7 mg/kg. Particular illustrative dosing ranges include for example, from about 0.1 mg/kg to about 10 mg/kg, or from about 0.2 mg/kg to about 7 mg/kg or from about 0.2 mg/kg to less than about 0.7 mg/kg.

Anti-Androgen

Embodiments of the present invention relate to anti-androgens, or a pharmaceutically acceptable salt thereof.

In one embodiment, the anti-androgen, or a pharmaceutically acceptable salt thereof, is a compound which degrades the androgen receptor.

In one embodiment, the anti-androgen, or a pharmaceutically acceptable salt thereof, is a compound which inhibits and/or suppresses the production of androgens.

In one embodiment, the anti-androgen is abiraterone, or a pharmaceutically acceptable salt thereof, such as abiraterone acetate (marketed as Zytiga™), a steroidal CY17A1 inhibitor which is disclosed in U.S. Pat. No. 5,604,213 which published on 18 Feb. 1997, the contents of which are incorporated herein by reference.

In one embodiment the anti-androgen, or a pharmaceutically acceptable salt thereof, is an androgen receptor inhibitor, or a pharmaceutically acceptable salt thereof. Androgen receptor inhibitors include, but are not limited to, non-steroidal small molecule androgen-receptor inhibitors, or pharmaceutically acceptable salts thereof. Androgen receptor inhibitors can be determined by methods known to those of skilled in the art, for example using in vitro assays and/or cellular ligand binding assays and/or gene expression assays such as those disclosed in Tran C., et al., Science, 2009, 324, 787-790.

Examples of specific androgen receptor inhibitors that are useful in the present invention include those disclosed in International patent application PCT/US2006/011417, which published on 23 Nov. 2006 as WO 2006/124118, the contents of which are included herein by reference, or a pharmaceutically acceptable salt thereof. Specific androgen receptor inhibitors disclosed therein useful as the androgen receptor inhibitor for the present invention include, but are not limited to, androgen receptor inhibitors selected from the group consisting of:

RD7; RD8; RD10; RD35; RD36; RD37; RD57; RD58; RD90; RD91; RD92; RD93; RD94; RD95; RD96; RD97; RD100; RD102; RD119; RD120; RD130; RD131; RD145; RD152; RD153; RD163; RD162; RD162′; RD162″; RD168; RD169; and RD170

or a pharmaceutically acceptable salt thereof.

Other examples of specific androgen receptor inhibitors that are useful in the present invention include those disclosed in International patent application PCT/US2007/007854, which published on 8 Nov. 2007 as WO 2007/127010, the contents of which are included herein by reference, or a pharmaceutically acceptable salt thereof.

Other examples of specific androgen receptor inhibitors that are useful in the present invention include those disclosed in International patent application PCT/US2008/012149, which published on 30 Apr. 2009 as WO 2009/055053, the contents of which are included herein by reference, or a pharmaceutically acceptable salt thereof.

Other examples of specific androgen receptor inhibitors that are useful in the present invention include those disclosed in International patent application PCT/US2007/007485, which published on 8 Nov. 2007 as WO 2007/126765, the contents of which are included herein by reference. Examples of specific androgen receptor inhibitors disclosed therein useful as the androgen receptor inhibitor for the present invention include, but are not limited to, androgen receptor inhibitors selected from the group consisting of:

A51; and A52

or a pharmaceutically acceptable salt thereof.

Other examples of specific androgen receptor inhibitors that are useful in the present invention include those disclosed in International patent application PCT/US2010/030581, which published on 14 Oct. 2010 as WO 2010/118354, the contents of which are included herein by reference, or a pharmaceutically acceptable salt thereof.

Other examples of specific androgen receptor inhibitors that are useful in the present invention include those disclosed in International patent application PCT/US2010/051770, which published on 14 Apr. 2011 as WO 2011/044327, the contents of which are included herein by reference, or a pharmaceutically acceptable salt thereof.

Other examples of specific androgen receptor inhibitors that are useful in the present invention include those disclosed in International patent application PCT/US2010/025283, which published on 2 Sep. 2010 as WO 2010/099238, the contents of which are included herein by reference. Examples of specific androgen receptor inhibitors disclosed therein useful as the androgen receptor inhibitor for the present invention include, but are not limited to, androgen receptor inhibitors selected from the group consisting of:

MII

or a pharmaceutically acceptable salt thereof.

Other examples of specific androgen receptor inhibitors that are useful in the present disclosure include those disclosed in International patent application PCT/FI2010/000065, which published on 5 May 2011 as WO 2011/051540, the contents of which are included herein by reference.

Other examples of specific androgen receptor inhibitors that are useful in the present disclosure include those disclosed in U.S. Pat. No. 4,636,505, published on 13 Jan. 1987, the contents of which are included herein by reference.

In one embodiment, the androgen receptor inhibitor useful in the present disclosure is enzalutamide:

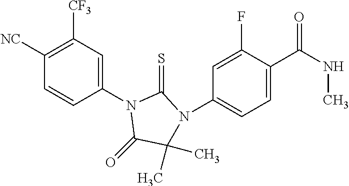 [SMILES: CNC(=O)c1ccc(N2C(=S)N(c3ccc(C#N)c(C(F)(F)F)c3)C(=O)C2(C)C)cc1F]

or a pharmaceutically acceptable salt thereof, also known as RD162′; 4-[3-[4-cyano-3-(trifluoromethyl)phenyl]-5,5-dimethyl-4-oxo-2-thioxo-1-imidazolidinyl]-2-fluoro-N-methyl-benzamide; or 4-{3-[4-cyano-3-(trifluoromethyl)phenyl]-5,5-dimethyl-4-oxo-2-sulfanylideneimidazolidin-1-yl}-2-fluoro-N-methylbenzamide; which is disclosed in PCT/US2006/011417, which published on 23 Nov. 2006 as WO 2006/124118, the contents of which are included herein by reference.

In one embodiment, the androgen receptor inhibitor useful in the present invention is N-desmethyl enzalutamide:

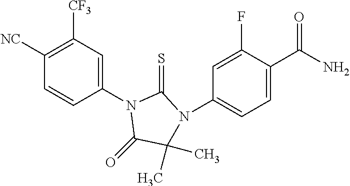 [SMILES: CC1(C)C(=O)N(c2ccc(C#N)c(C(F)(F)F)c2)C(=S)N1c1ccc(C(N)=O)c(F)c1]

or a pharmaceutically acceptable salt thereof, also known as 4-[3-[4-cyano-3-(trifluoromethyl)phenyl]-5,5-dimethyl-4-oxo-2-thioxoimidazolidin-1-yl]-2-fluorobenzamide; or MII; which is disclosed in PCT/US2010/025283, which published on 2 Sep. 2010 as WO 2010/099238, the contents of which are included herein by reference.

In one embodiment, the androgen receptor inhibitor useful in the present invention is apalutamide:

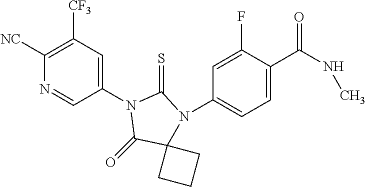 [SMILES: CNC(=O)c1ccc(N2C(=S)N(c3cnc(C#N)c(C(F)(F)F)c3)C(=O)C23CCC3)cc1F]

or a pharmaceutically acceptable salt thereof, also known as ARN-509; or 4-{7-[6-cyano-5-(trifluoromethyl)pyridine-3-yl]-8-oxo-6-thioxo-5,7-diazaspiro[3,4]octan-5yl}-2-fluoro-N-methylbenzamide; which is disclosed in PCT/US2007/007485, which published on 8 Nov. 2007 as WO 2007/126765, the contents of which are included herein by reference. In one embodiment, the androgen receptor inhibitor useful in the present invention is a pharmacologically active metabolite of apalutamide, or a pharmaceutically acceptable salt thereof.

In one embodiment, the androgen receptor inhibitor useful in the present invention is darolutamide:

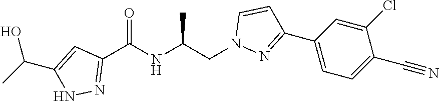 [SMILES: CC(O)c1cc(C(=O)N[C@@H](C)Cn2ccc(-c3ccc(C#N)c(Cl)c3)n2)n[nH]1]

or a pharmaceutically acceptable salt thereof, also known as N-[(2S)-1-[3-(3-chloro-4-cyanophenyl)-1H-pyrazol-1-yl]propan-2-yl]-5-(1-hydroxyethyl)-1H-pyrazole-3-carboxamide which is disclosed in PCT/FI2010/000065, which published on 5th May 2011 as WO 2011/051540, the contents of which are included herein by reference.

In one embodiment, the androgen receptor inhibitor useful in the present invention is bicalutamide:

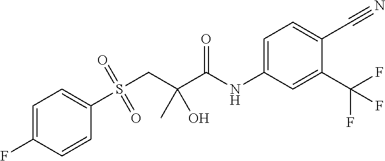 [SMILES: CC(O)(CS(=O)(=O)c1ccc(F)cc1)C(=O)Nc1ccc(C#N)c(C(F)(F)F)c1]

or a pharmaceutically acceptable salt thereof, marketed as Casodex™, which is disclosed in U.S. Pat. No. 4,636,505, published on 13 Jan. 1987, the contents of which are included herein by reference.

In one embodiment, the androgen receptor inhibitor useful in the present invention is deuterated enzalutamide, HC-1119:

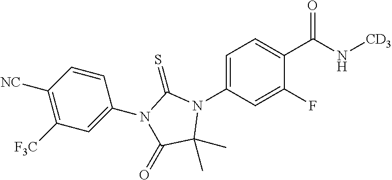 [SMILES: [2H]C([2H])([2H])NC(=O)c1ccc(N2C(=S)N(c3ccc(C#N)c(C(F)(F)F)c3)C(=O)C2(C)C)cc1F]

or a pharmaceutically acceptable salt thereof, also known as 4-{7-[4-cyano-3-(trifluoromethyl)phenyl]-5,5-dimethyl-4-oxo-2-thio-1-imidazolidinyl}-2-fluoro-N-trideuteromethylbenzamide which is disclosed in PCT/CN2012/086573, which published on 20 Jun. 2013 as WO2013/087004, the contents of which are induced herein by reference.

In one embodiment, the androgen receptor inhibitor useful in the present invention is proxalutamide:

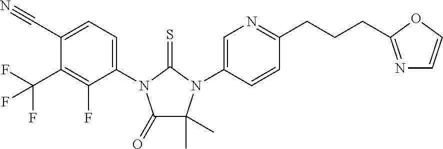 [SMILES: CC1(C)C(=O)N(c2ccc(C#N)c(C(F)(F)F)c2F)C(=S)N1c1ccc(CCCc2ncco2)nc1]

or a pharmaceutically acceptable salt thereof, also known as 4-(4,4-dimethyl-3-(6-(3-(oxazol-2-yl)propyl)pyridin-3-yl)-5-oxo-2-thioxoimidazolidin-1-yl)-3-fluoro-2-(trifluoromethyl)benzonitrile which is disclosed in PCT/CN2012/072091, which published on 13 Sep. 2012 as WO2012/119559, the contents of which are induced herein by reference.

In one embodiment, the androgen receptor inhibitor useful in the present invention is nilutamide, or a pharmaceutically acceptable salt thereof.

In one embodiment, the androgen receptor inhibitor useful in the present invention is flutamide, or a pharmaceutically acceptable salt thereof.

Preferred androgen receptor inhibitors useful for the present invention are selected from the group consisting of enzalutamide; N-desmethyl enzalutamide; darolutamide; and apalutamide; or a pharmaceutically acceptable salt thereof.

More preferred androgen receptors inhibitors useful for the present invention is enzalutamide, or a pharmaceutically acceptable salt thereof. More preferably the androgen receptor inhibitor is enzalutamide.

In one embodiment the anti-androgen, or a pharmaceutically acceptable salt thereof, is administered in combination with androgen deprivation therapy.

In one embodiment the anti-androgen, or a pharmaceutically acceptable salt thereof, is administered in combination with androgen deprivation therapy, which androgen deprivation therapy is selected from the group consisting of a luteinizing hormone-releasing hormone (LHRH) agonist, a LHRH antagonist, a gonadotropin releasing hormone (GnRH) agonist and a GnRH antagonist.

In one embodiment the androgen deprivation therapy is selected from the group consisting of leuprolide (also known as leuprorelin, for example Lupron or Eligardor Viadur and the like); buserelin (for example Suprefact); gonadorelin; goserelin (for example Zoladex); histrelin (for example Vantas); nafarelin; triptorelin (for example Trelstar); deslorelin; fertirelin; abarelix (for example Plenaxis); cetrorelix; degarelix (for example Firmagon); ganirelix; ozarelix; elagolix (for example Orilissa); relugolix; and linzagolix.

In one embodiment the androgen deprivation therapy is leuprolide.

In one embodiment the androgen deprivation therapy is goserelin.

In one embodiment the androgen deprivation therapy is degarelix.

In one embodiment the anti-androgen is enzalutamide and the androgen deprivation therapy is selected from the group consisting of leuprolide; buserelin gonadorelin; goserelin; histrelin; nafarelin; triptorelin; deslorelin; fertirelin; abarelix; cetrorelix; degarelix; ganirelix; ozarelix; elagolix; relugolix; and linzagolix. In one embodiment the anti-androgen is enzalutamide and the androgen deprivation therapy is selected from the group consisting of leuprolide, goserelin and degarelix.

In one embodiment the anti-androgen is N-desmethyl enzalutamide and the androgen deprivation therapy is selected from the group consisting of leuprolide; buserelin gonadorelin; goserelin; histrelin; nafarelin; triptorelin; deslorelin; fertirelin; abarelix; cetrorelix; degarelix; ganirelix; ozarelix; elagolix; relugolix; and linzagolix. In one embodiment the anti-androgen is N-desmethyl enzalutamide and the androgen deprivation therapy is selected from the group consisting of leuprolide, goserelin and degarelix.

In one embodiment the anti-androgen is apalutamide and the androgen deprivation therapy is selected from the group consisting of leuprolide; buserelin gonadorelin; goserelin; histrelin; nafarelin; triptorelin; deslorelin; fertirelin; abarelix; cetrorelix; degarelix; ganirelix; ozarelix; elagolix; relugolix; and linzagolix. In one embodiment the anti-androgen is apalutamide and the androgen deprivation therapy is selected from the group consisting of leuprolide, goserelin and degarelix.

In one embodiment the anti-androgen is abiraterone, preferably abiraterone acetate, and the androgen deprivation therapy is selected from the group consisting of leuprolide; buserelin gonadorelin; goserelin; histrelin; nafarelin; triptorelin; deslorelin; fertirelin; abarelix; cetrorelix; degarelix; ganirelix; ozarelix; elagolix; relugolix; and linzagolix. In one embodiment the anti-androgen is abiraterone, preferably abiraterone acetate, and the androgen deprivation therapy is selected from the group consisting of leuprolide, goserelin and degarelix.

Unless indicated otherwise, all references herein to the anti-androgens and androgen receptor inhibitors includes references to salts, solvates, hydrates and complexes thereof, and to solvates, hydrates and complexes of salts thereof, including polymorphs, stereoisomers, and isotopically labeled versions thereof.

In accordance with the methods described herein, an effective amount of an anti-androgen, or pharmaceutically acceptable salt thereof, may be administered. One of ordinary skill in the art can determine how much of the anti-androgen is sufficient to provide clinically relevant inhibition. For example, one of ordinary skill in the art can refer to the literature and/or administer a series of increasing amounts of the anti-androgen, or pharmaceutically acceptable salt thereof, to determine which amount or amounts provide clinically relevant activity.

Treatment

In certain embodiments, the subject has received one, two, three, four, five or more prior cancer treatments. In other embodiments, the subject is treatment-naïve. In some embodiments, the subject has progressed on other cancer treatments. In certain embodiments, the prior cancer treatment comprised an immunotherapy. In other embodiments, the prior cancer treatment comprised a chemotherapy. In some embodiments, the tumor has reoccurred. In some embodiments, the tumor is metastatic. In other embodiments, the tumor is not metastatic.

In some embodiments, the subject has received a prior therapy to treat the tumor and the tumor is relapsed or refractory. In some embodiments, the subject has received a prior immuno-oncology therapy to treat the tumor and the tumor is relapsed or refractory. In some embodiments, the subject has received more than one prior therapy to treat the tumor and the subject is relapsed or refractory.

The treatment methods described herein can continue for as long as the clinician overseeing the patient's care deems the treatment method to be effective. Non-limiting parameters that indicate the treatment method is effective include any one or more of the following: tumor shrinkage (in terms of weight and/or volume); a decrease in the number of individual tumor colonies; tumor elimination; and progression-free survival. Change in tumor size may be determined by any suitable method such as imaging. Various diagnostic imaging modalities can be employed, such as computed tomography (CT scan), dual energy CDT, positron emission tomography and MRI.

Based upon the long acting nature of RSLAIL-2, such composition may be administered relatively infrequently (e.g., once every three weeks, once every two weeks, once every 8-10 days, once every week, etc.).

Exemplary lengths of time associated with the course of therapy include about one week; about two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years and about five years.

Administration, may be oral or parenteral. Other modes of administration are also contemplated, such as pulmonary, nasal, buccal, rectal, sublingual and transdermal. As used herein, the term “parenteral” includes subcutaneous, intravenous, intra-arterial, intratumoral, intralymphatic, intraperitoneal, intracardiac, intrathecal, and intramuscular injection, as well as infusion injections. An agent being administered parenterally typically is given as a composition comprising a diluent. With respect to possible diluents, the diluent can be selected from the group consisting of bacteriostatic water for injection, dextrose 5% in water, phosphate-buffered saline, Ringer's solution, lactated Ringer's solution, saline, sterile water, deionized water, and combinations thereof. One of ordinary skill in the art can determine through routing testing whether two given pharmacological components are compatible together in a given formulation.

The presently described combinations and methods can be used to treat a patient suffering from any condition that can be remedied or prevented by the methods provided herein, such as cancer. Exemplary conditions are cancers, such as, for example, head and neck cancer (including metastatic and recurring), fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, brain cancer, breast cancer, ovarian cancer, prostate cancer (including metastatic castration-resistant prostate cancer), squamous cell cancer, basal cell cancer, adenocarcinoma, sweat gland cancer, sebaceous gland cancer, papillary cancer, papillary adenocarcinomas, cystadenocarcinoma, medullary cancer, bronchogenic cancer, renal cell cancer, hepatoma, bile duct cancer, choriocarcinoma, seminoma, embryonal cancer, Wilms' tumor, cervical cancer, Hodgkin lymphoma, non-Hodgkin lymphoma, testicular cancer, lung cancer, small cell lung cancer, brain cancer, bladder cancer, epithelial cancer, glioma, astrocytoma, medulloblastoma, craniopharyngioma, ependymoma, pinealoma, hemangioblastoma, acoustic neuroma, oligodendroglioma, meningioma, melanoma, multiple myeloma, neuroblastoma, retinoblastoma and leukemias. In some particular embodiments, the cancer to be treated is a solid cancer, such as for example, head and neck cancer (including metastatic and recurring), breast cancer, ovarian cancer, colon cancer, prostate cancer (including metastatic castration-resistant prostate cancer), bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, maxillary sinus cancer, bladder cancer, esophageal cancer, Hodgkin's disease and adrenocortical cancer. In some particular embodiments, the cancer to be treated is prostate cancer. In some particular embodiments, the cancer to be treated is castration resistant prostate cancer. In some particular embodiments, the cancer to be treated is metastatic castration resistant prostate cancer.

Administration of compounds of the invention may be effected by any method that enables delivery of the compounds to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, a therapeutic agent of the combination therapy of the present invention may be administered as a single bolus, as several divided doses administered over time, or the dose may be proportionally reduced or increased as indicated by the exigencies of the therapeutic situation. It may be particularly advantageous to formulate a therapeutic agent in a dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention may be dictated by and directly dependent on (a) the unique characteristics of the therapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose may be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, taking into consideration factors such as the severity of the disorder or condition, the rate of administration, the disposition of the compound and the discretion of the prescribing physician. The dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the therapeutic agent are well-known in the relevant art and would be understood to be encompassed by the skilled artisan once provided the teachings disclosed herein.

In some embodiments, at least one of the therapeutic agents in the combination therapy is administered using the same dosage regimen (dose, frequency and duration of treatment) that is typically employed when the agent is used as a monotherapy for treating the same cancer. In other embodiments, the subject received a lower total amount of at least one of the therapeutic agents in the combination therapy than when the same agent is used as a monotherapy, for example a lower dose of therapeutic agent, a reduced frequency of dosing and/or a shorter duration of dosing. An effective dosage of an anti-androgen, or a pharmaceutically acceptable salt thereof, is in the range of from about 0.001 to about 100 mg per kg body weight per day, preferably about 1 to about 35 mg/kg/day, in single or divided doses. For a 70 kg human, this would amount to about 0.01 to about 7 g/day, preferably about 0.02 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

An effective dosage of an androgen receptor inhibitor, or a pharmaceutically acceptable salt thereof, is in the range of from about 0.001 to about 100 mg per kg body weight per day, preferably about 1 to about 35 mg/kg/day, in single or divided doses. For a 70 kg human, this would amount to about 0.01 to about 7 g/day, preferably about 0.02 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

In one embodiment the androgen receptor inhibitor is enzalutamide, which enzalutamide is dosed in accordance with the approved label with a daily dose of 160 mg once daily. In one embodiment the androgen receptor inhibitor is enzalutamide, which enzalutamide is dosed with a daily dose of 120 mg once daily. In one embodiment the androgen receptor inhibitor is enzalutamide, which enzalutamide is dosed with a daily dose of 80 mg once daily. Dosage adjustments of enzalutamide, in accordance with full prescribing information may be readily determined by one of ordinary skill in the art, such as if the enzalutamide is to be dosed in concomitantly with a strong CYP2C8 inhibitor then the dose of enzalutamide should be reduced in accordance with the full prescribing information, such as to 80 mg once daily; or alternatively if the enzalutamide is to be dosed concomitantly with a CYP3A4 inducer then the dose of enzalutamide should be increased in accordance with the full prescribing information, such as to 240 mg daily, as can be determined by one of ordinary skill in the art.

In an embodiment the anti-androgen is abiraterone acetate, which abiraterone acetate is dosed in accordance with the approved label with a daily dose of 1000 mg once daily in combination with prednisone 5 mg twice daily. Dosage adjustments of abiraterone acetate, in accordance with full prescribing information may be readily determined by one of ordinary skill in the art, such as if the abiraterone acetate is to be dosed concomitantly with a strong CYP3A4 inducer, then the dosage of abiraterone acetate may need to be increased for example to 1000 mg twice per day; if the abiraterone acetate is to be dosed concomitantly with a CYP2D6 substrate, then the dosage of abiraterone acetate may need to be reduced; if the abiraterone acetate is to be dosed to a subject or subject with baseline moderate hepatic impairment then the dose may need to be reduced, such as to 250 mg once daily; if the abiraterone acetate is to be dosed to a subject or subject who develops hepatotoxicity then the dose may need to be reduced, such as to 750 mg or 500 mg once daily.

Repetition of the administration or dosing regimens, or adjustment of the administration or dosing regimen may be conducted as necessary to achieve the desired treatment. A “continuous dosing schedule” as used herein is an administration or dosing regimen without dose interruptions, e.g. without days off treatment. Repetition of 21 or 28 day treatment cycles without dose interruptions between the treatment cycles is an example of a continuous dosing schedule. In an embodiment, the compounds of the combination of the present invention can be administered in a continuous dosing schedule.

### Kits

The therapeutic agents of the combination therapies of the present invention may conveniently be combined in the form of a kit suitable for coadministration of the compositions.

In one aspect, the present invention relates to a kit which comprises a first container, a second container, a third container and a package insert, wherein the first container comprises at least one dose of a PD-1 axis binding antagonist; the second container comprises at least one dose of a CD-122-biased cytokine agonist; the third container comprises at least one dose of an anti-androgen, or a pharmaceutically acceptable salt thereof, or and the package insert comprises instructions for treating a subject for cancer using the medicaments.

In one embodiment, the kit of the present invention may comprise one or more of the active agents in the form of a pharmaceutical composition, which pharmaceutical composition comprises an active agent, or a pharmaceutically acceptable salt or solvate thereof, and a pharmaceutically acceptable carrier. The kit may contain means for separately retaining said compositions, such as a container, divided bottle, or divided foil packet. An example of such a kit is the familiar blister pack used for the packaging of tablets, capsules and the like.

The kit may be particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid. The kit may further comprise other materials that may be useful in administering the medicaments, such as diluents, filters, IV bags and lines, needles and syringes, and the like.

### Further Therapeutic Agents

In a further aspect, the methods and combination therapies of the present invention may additionally comprise administering further anti-cancer agents, such as anti-tumor agents, anti-angiogenesis agents, signal transduction inhibitors and antiproliferative agents, which amounts are together effective in treating said cancer. In some such embodiments, the anti-tumor agent is selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, androgen deprivation therapy and anti-androgens.

In one embodiment of the methods and combination therapies of the present invention, the regimen includes a further active agent, wherein the further active agent is androgen deprivation therapy, such as an luteinizing hormone-releasing hormone (LHRH) agonist, an LHRH antagonist, or a gonadotropin-releasing hormone (GnRH) agonist or GnRH antagonist, including, but not limited to, leuprolide, buserelin, nafarelin, histrelin, goserelin, or deslorelin, and the like.

In one embodiment of the methods and combination therapies of the present invention, the regimen includes a further active agent, wherein the further active agent is androgen deprivation therapy, such as an LHRH agonist and the like.

In one embodiment the androgen deprivation therapy is a LHRH agonist.

In one embodiment the androgen deprivation therapy is a LHRH antagonist.

In one embodiment the androgen deprivation therapy is a GnRH agonist.

In one embodiment the androgen deprivation therapy is a GnRH antagonist.

In one embodiment the androgen deprivation therapy is selected from the group consisting of leuprolide (also known as leuprorelin, for example Lupron or Eligardor Viadur and the like); buserelin (for example Suprefact); gonadorelin; goserelin (for example Zoladex); histrelin (for example Vantas); nafarelin; triptorelin (for example Trelstar); deslorelin; fertirelin; abarelix (for example Plenaxis); cetrorelix; degarelix (for example Firmagon); ganirelix; ozarelix; elagolix (for example Orilissa); relugolix; and linzagolix.

In one embodiment the androgen deprivation therapy is selected from the group consisting of leuprolide, goserelin and degaralix.

In one embodiment the androgen deprivation therapy is leuprolide. In some embodiments the leuprolide is administered intramuscularly at a dose of about 7.5 mg every month, or about 22.5 mg every three months, or about 30 mg every four months.

In one embodiment the androgen deprivation therapy is leuprolide. In some embodiments the leuprolide is administered subcutaneously at a dose of about 7.5 mg every month, or about 22.5 mg every three months, or about 30 mg every four months, or about 45 mg every six months, or about 65 mg every 12 months.

In one embodiment the androgen deprivation therapy is goserelin. In some embodiments the goserelin is administered subcutaneously at a dose of about 3.6 mg every month, or about 10.8 mg every three months.

In one embodiment the androgen deprivation therapy is degarelix. In some embodiments the degarelix is administered intramuscularly at an initial dose of about 240 mg, which initial dose may be optionally divided into several smaller doses, for example 2 doses of about 120 mg, followed by a maintenance dose of about 80 mg every month.

All articles, books, patents, patent publications and other publications referenced herein are incorporated by reference in their entireties. In the event of an inconsistency between the teachings of this specification and the art incorporated by reference, the meaning of the teachings and definitions in this specification shall prevail (particularly with respect to terms used in the claims appended herein). For example, where the present application and a publication incorporated by reference defines the same term differently, the definition of the term shall be preserved within the teachings of the document from which the definition is located.

### EXAMPLES

### Example 1

Phase 1b and 2 study of triple combination of avelumab, bempegaldesleukin, and enzalutamide in patients with metastatic castration resistant prostate cancer (mCRPC). Different cohorts of the study are described in below Table 1.

TABLE 1
Phase 1b and Phase 2 Study Design Scheme

| Phase 1b | Phase 2 |
|---|---|
| Dosing Finding Cohorts | Expansion Cohorts |
| A1: SCCHN | |
| Avelumab | |
| Bempegaldesleukin | |
| (Combination A) | |
| C1: mCRPC | C2: mCRPC |
| Avelumab | Avelumab |
| Enzalutimde | Enzalutamide |
| Bempegaldesleukin | Bempegaldesleukin |
| (Combination C) | (Combination C) |

Phase 1b of Combination A (cohort A1): This is a dose finding study for avelumab and Bempegaldesleukin in first line SCCHN patients, with dosing levels shown in below Table 1A.

TABLE 1A
Dosing levels of Avelumab and Bempegaldesleukin combination

| | Avelumab dose | Bempegaldesleukin |
|---|---|---|
| Dose | IV | dose IV |
| Level | (mg Q2W) | (mg/kg Q2W) |
| D0 | 800 | 0.006 |
| D-1 | 800 | 0.003 |

Phase 1b of Combination C (Cohort C1):

This will be a dose finding study for avelumab in combination with bempegaldesleukin and enzalutamide (Combination C) in patients with mCRPC. Table 1C describes the planned dose levels for the combination.

Primary objectives: To assess the dose-limiting toxicity (DLT) rate of avelumab in combination with bempegaldesleukin and enzalutamide in patients with mCRPC in order to determine the recommended Phase 2 dose (RP2D) for the combination.

TABLE 1C
Phase 1b of Combination C (cohort C1) Avelumab, Bempegaldesleukin,
and Enzalutamide Dose Levels

| | Avelumab dose | Bempegaldesleukin | Enzalutamide |
|---|---|---|---|
| Dose | IV | dose IV | dose oral (mg |
| Level | (mg Q2W) | (mg/kg Q2W) | QD) |
| D0-A | 800 | 0.006 | 160 |
| D-1A | 800 | 0.006 | 120 |
| D-2A | 800 | 0.006 | 80 |
| D0B | 800 | 0.003 | 160 |
| D-1B | 800 | 0.003 | 120 |
| D-2B | 800 | 0.003 | 80 |

Guidance for phase 1b dosing and enrollment decisions for both Combination A and Combination B will be based on a Bayesian Logistic Regression Model (BLRM) and will incorporate single agent and available double agent dose limiting toxicity (DLT) data (historical and prospectively across dose combinations) to estimate the posterior probability of under-dosing, target dosing and overdosing.

Beginning with the starting dose level, cohorts of 3-6 patients will be enrolled, treated, and monitored during the 28 days DLT evaluation period (cycle 1). Patients who withdraw from study treatment before receiving at least 2 doses of bempegaldesleukin and avelumab (Combination A) and at least 75% of the planned dose of enzalutamide (Combination C) in Cycle 1 for reasons other than treatment-related toxicity are not evaluable for DLT. A minimum of 3 DLT-evaluable patients from each cohort is required. Additional patients will be enrolled in the specific enrollment cohort to replace patients who are not considered DLT-evaluable.

The posterior distributions will be summarized to provide the posterior probability that the risk of DLT lie within the intervals shown below:
Underdosing: [0, 0.16]
Target toxicity: [0.16, 0.33]
Excessive toxicity or overdosing: [0.33, 1]
   A dose level combination is a potential candidate for being the maximum tolerated dose (MTD) level when all the following criteria are net:
6 or more participants have been treated at that dose;
Probability of target dosing is more than 0.50;
Probability of overdosing is less than 0.25

An RP2D below the MTD may be determined based on other safety, clinical activity, PK and pharmacodynamic (PD) data. Nine DLT-evaluable participants are needed to be treated at RP2D if no DLT is observed, and 12 evaluable participants if at least 1 DLT is observed.

Phase 2 of Combination C (Cohort C2):

The RP2D dose level of the avelumab, bempegaldesleukin and enzalutamide combination (Combination C) in mCRPCwill be chosen based on the corresponding phase 1 b study of both Combination A (cohort A1) and Combination C (cohorts C1), for further clinical development and for evaluation in the Phase 2 part of the study; a RP2D below the MTD may be identified based on other safety, clinical, PK, and PD data.

Primary Objectives (cohort C2): To assess the prostate specific antigen (PSA) response rate of avelumab in combination with bempegaldesleukin and enzalutamide in patients with mCRPC after progression on abiraterone.

Primary endpoints (cohort C2): confirmed PSA response decrease 50% from base line confirmed by a second consecutive assessment at least 3 weeks later.

Secondary objectives (cohort C2): To assess the overall safety and tolerability of the combination C; and to assess other measures of anti-tumor activity; to characterize the PK of avelumab, bempegaldesleukin and enzalutamide; and assess immunogenicity of avelumab and bempegaldesleukin when combined with enzalutamide.

Secondary endpoints (cohort C2):
Confirmed soft tissue objective response (OR) as assessed by the investigator using RECIST v1.1 with no evidence of confirmed bone disease progression on repeat bone scan at least 6 weeks later per Prostate Cancer Working Group 3 (PCWG3) criteria.
Time to event endpoints as assessed by the investigator, using RECIST v1.1 and PCWG3 (bone disease), including time to tumor response (TTR), duration of response (DR), and progression free survival (PFS). Additional time-to-event endpoints include overall survival (OS) for all patients and time to prostate-specific antigen (PSA) progression (≥25% increase) for mCRPC patients.
Time to PSA progression (TTPSAP) defined according to the consensus guidelines of the PCWG3 criteria
Circulation tumor cells (CTC) count conversion (decrease from 5 or above CTC per 7.5 mL of blood at based line to less than 5 CTC per 7.5 mL of blood at at any assessment on treatment), and CTCO (decrease from CTC count of 1 or above CTC of 7.5 mL blood at baseline to 0 CTC per 7.5 mL of blood at any assessment on treatment)
PK parameters including trough concentrations (C_{trough}) for avelumab, bempegaldesleukin, and enzalutamide and N-desmethyl-enzalutamide and maximum concentrations (Cmax) for avelumab and bempegaldesleukin.
Anti-drug antibody (ADA) and neutralizing antibody (Nab) against avelumab and bempegaldesleukin and IL-2 when combined with enzalutamide.

Patient selection criteria (Combination C, cohort C1 and C2):
Patient must have progressed on 1 line of abiraterone acetate/prednisone anti-androgen therapy for treatment of mCRPC.
Patient must not have had prior chemotherapy for the treatment of mCRPC. Prior treatment with radium 223 or sipuleucel-T is allowed and it does not count for a line of prior chemotherapy regimen.

Avelumab Administration:

Avelumab will be administered as a 1 hour (or 50 to 70 minutes) IV infusion starting after bempegaldesleukin and enzalutamide is administered on day 1 and day 15 of each of the 28 days cycle. After cycle 1 day 1, avelumab can be administered up to 2 days before or after the scheduled treatment day of each cycle for administrative reasons. Within the 2-day window, avelumab and bempegaldesleukin should be administered on the same day, unless one treatment needs to be delayed or withheld due to toxicity reasons.

In order to mitigate infusion related reactions (IRRs) associated with avelumab, premedication with an antihistamine and with paracetamol (acetaminophen) 30 to 60 minutes prior to the first 4 infusions of avelumab is mandatory. Premedication is not mandatory beyond the first four infusions but should be administered for subsequent avelumab doses based on clinical judgment and presence/severity of prior infusion reactions. The premedication regimen may be modified based on local treatment standards and guidelines, as appropriate, provided it does not include systemic corticosteroids.

Bempegaldesleukin Administration:

bempegaldesleukin will be administered over 30 (+/−5) minutes every 2 weeks (+/−2 days). Within the 2-day window, avelumab and bempegaldesleukin should be administered on the same day, unless one treatment needs to be delayed or withheld due to toxicity reasons.

Enzalutamide Administration

Enzalutamide will be taken once daily starting on day 1 of cycle 1 and treatment should continue until end of treatment or withdrawal. On the day when the patient returns to the clinic for avelumab infusion and bempegaldesleukin infusion, enzalutamide will be taken at the clinic before or after the avelumab and bempegaldesleukin infusions.

1. A method of treating a subject having cancer comprising administering to the subject:
(i) an amount of a PD-1 axis binding antagonist;
(ii) an amount of a CD-122-biased cytokine agonist; and
(iii) an amount of an anti-androgen or a pharmaceutically acceptable salt thereof, wherein the amounts together are effective in treating cancer.

2. The method according to claim 1, wherein the subject is a mammal.

3. The method according to claim 1 wherein the subject is a human.

4. The method according to claim 1, wherein the cancer is prostate cancer.

5. The method according to claim 1, wherein the cancer is prostate cancer, which prostate cancer is metastatic.

6. The method according to claim 1, wherein the cancer is prostate cancer, which prostate cancer is metastatic castration resistant prostate cancer (mCRPC).

7. The method according to claim 1, wherein the anti-androgen, or a pharmaceutically acceptable salt thereof, is an androgen receptor inhibitor, or a pharmaceutically acceptable salt thereof.

8. The method according to claim 1, wherein the anti-androgen, or a pharmaceutically acceptable salt thereof, is enzalutamide, or a pharmaceutically acceptable salt thereof.

9. The method according to claim 1, wherein the anti-androgen is enzalutamide.

10. The method according to claim 1, wherein the PD-1 axis binding antagonist is avelumab.

11. The method according to claim 1, wherein the CD-122-biased cytokine agonist is bempegaldesleukin.

12. The method according to claim 1, wherein the PD-1 axis binding antagonist is avelumab and is administered in an IV dose of 800 mg Q2W, the anti-androgen is enzalutamide and is administered in an oral dose of 160 mg QD, the CD-122-biased cytokine agonist is bempegaldesleukin and is administered as an IV dose of about 0.003 mg/kg to 0.006 mg/kg Q2W, and wherein the cancer is mCRPC.

13. The method of claim 1, wherein the cancer is mCRPC, and the subject having cancer has progressed on 1 line of abiraterone acetate/prednisone anti-androgen therapy for treatment of mCRPC.

14. The method of claim 1, wherein the cancer is mCRPC, and the subject having cancer has had bilateral orchiectomy.

15. The method of claim 1 wherein the cancer is mCRPC, and the subject having cancer is being treated with androgen deprivation therapy.

16. The method of claim 15 wherein the androgen deprivation therapy is selected from the group consisting of a gonadotropin releasing hormone (GnRH) agonist and a gonadotropin releasing hormone (GnRH) antagonist.

17. A combination of a PD-1 axis binding antagonist, a CD-122-biased cytokine agonist, and an anti-androgen, or a pharmaceutically acceptable salt thereof.

18-20. (canceled)

21. The use of a PD-1 axis binding antagonist, a CD-122-biased cytokine agonist, and an anti-androgen, or a pharmaceutically acceptable salt thereof in the manufacture of a medicament for the treatment of cancer in a subject.


SEQUENCE LISTING (1 sequence)

SEQ ID NO: 1 - protein, 288 aa, Homo sapiens
MRIFAVFIFMTYWHLLNAFTVTVPKDLYWEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILWDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET